(19) United States
(12) Patent Application Publication
(10) Pub. No.: US 2020/0321118 A1
(43) Pub. Date: Oct. 8, 2020

(54) METHOD FOR DOMAIN ADAPTATION BASED ON ADVERSARIAL LEARNING AND APPARATUS THEREOF

(71) Applicant: Lunit Inc., Seoul (KR)

(72) Inventors: Hyo-Eun KIM, Seoul (KR); HyunJae LEE, Seoul (KR)

(21) Appl. No.: 16/698,878

(22) Filed: Nov. 27, 2019

(30) Foreign Application Priority Data
Apr. 2, 2019 (KR) 10-2019-0038197

(51) Int. Cl. G16H 50/20; G06N 20/00; G16H 30/20

(52) U.S. Cl. CPC G16H 50/20; G16H 30/20; G06N 20/00

(57) ABSTRACT

A domain adaptation method and apparatus based on adversarial learning are provided. The method may include extracting feature data from multiple data sets, training a first discriminator discriminating a domain for a first class using first feature data extracted from a first data set corresponding to a first class of a first domain among the multiple data sets and training the first discriminator using second feature data extracted from a second data set corresponding to the first class of a second domain among the multiple data sets. The method may also include training a second discriminator discriminating a domain for a second class using third feature data extracted from a third data set that corresponds to a second class of the first domain, and training the second discriminator using fourth feature data extracted from a fourth data set that corresponds to the second class of the second domain.

### CROSS-REFERENCE TO RELATED APPLICATION

This application claims priority to and the benefit of Korean Patent Application No. 2019-0038197, filed on Apr. 2, 2019, the disclosure of which is incorporated herein by reference in its entirety.

### BACKGROUND

### 1. Technical Field

This disclosure generally relates a method for domain adaptation based on adversarial learning and an apparatus thereof. More specifically, this disclosure generally relates a method of performing domain adaptation between a source domain and target domain while improving model performance of the target domain by adversarial learning and reducing cost of model construction, and an apparatus to support this method.

### 2. Discussion of Related Technology

In the field of machine learning, domain adaptation refers to a method of training a model so that source domain and target domain cannot be discriminated.

Domain adaptation can be utilized to reduce cost of model construction in the target domain. Otherwise, it can be used to construct a model that shows desirable performance in the target domain using the source domain that can easily secure massive data sets.

However, even if domain adaptation is performed on similar domains, it is difficult to construct a model that shows desirable performance in the target domain.

### SUMMARY

One inventive aspect is a method of performing domain adaptation based on adversarial learning by using neural network that includes a class-specific discriminator and an apparatus thereof.

Another aspect is, in relation to adversarial learning by neural network that includes multiple discriminators, a method and apparatus for utilizing discriminators that correspond to each of multiple classes included in domain and allow the neural network to learn better representation for performing target task.

Another aspect is, in relation to domain adaptation based on adversarial learning by neural network that includes multiple discriminators, a method and apparatus for improving performance of domain adaptation based on adversarial learning by adjusting learning based on inverted label of the discriminators.

Aspects of this disclosure are not limited to the above described aspects, and any other technical aspects not mentioned can be clearly understood by an ordinary person in the technical field of this disclosure according to the description below.

A method for domain adaptation based on adversarial learning according to some embodiments of this disclosure intended to solve the technical problems can comprise, for the method executed using a computing device, extracting feature data from multiple data sets, training first discriminator that discriminates domain of data corresponding to first class using first feature data extracted from first data set that corresponds to first class of first domain among the multiple data sets, training the first discriminator using second feature data extracted from second data set that corresponds to the first class of second domain among the multiple data sets, training second discriminator that discriminates domain of data corresponding to second class using third feature data extracted from third data set that corresponds to second class of the first domain among the multiple data sets, and training the second discriminator using fourth feature data extracted from fourth data set that corresponds to the second class of the second domain among the multiple data sets.

A domain adaptation apparatus based on adversarial learning according to some other embodiments of this disclosure intended to solve the technical problems can comprise a memory that stores one or more instructions and a processor that, by executing the stored one or more instructions, trains first discriminator to discriminate domain of data corresponding to first class using first feature data extracted from first data set of first domain among the multiple data sets, trains the first discriminator using second feature data extracted from second data set that corresponds to the first class of second domain among the multiple data sets, trains second discriminator to discriminate domain of data corresponding to second class using third feature data extracted from third data set that corresponds to second class of the first domain among the multiple data sets, and trains the second discriminator using fourth feature data extracted from fourth data set that corresponds to the second class of the second domain among the multiple data sets.

A computer program according to some other embodiments of this disclosure intended to solve the technical problems can be saved on a computer readable recording medium in order to execute, in combination with a computing device, extracting feature data from multiple data sets, training first discriminator to discriminate domain of data corresponding to first class using first feature data extracted from first data set that corresponds to first class of first domain among the multiple data sets, training the first discriminator using second feature data extracted from second data set that corresponds to the first class of second domain among the multiple data sets, training second discriminator to discriminate domain of data corresponding to second class using third feature data extracted from third set that corresponds to second class of the first domain among the multiple data sets, training the second discriminator using fourth feature data extracted from fourth data set that corresponds to the second class of the second domain among the multiple data sets.

### BRIEF DESCRIPTION OF THE DRAWINGS

These and/or other aspects will become apparent and more readily appreciated from the following description of embodiments, taken in conjunction with the accompanying drawings.

FIG. 1 and FIG. 2 are conceptual diagrams explaining a machine learning apparatus and learning environment according to some embodiments of this disclosure.

FIG. 3 is a flow diagram for a domain adaptation method based on adversarial learning according to some embodiments of this disclosure.

FIG. 4 is a flow diagram that illustrates detailed process of S100 acquiring data sets illustrated in FIG. 3.

FIG. 5 and FIG. 6 are conceptual diagrams further explaining a domain adaptation method based on adversarial learning according to some embodiments of this disclosure.

FIG. 7 is a flow diagram that illustrates detailed process of S500 learning output layer illustrated in FIG. 3.

FIG. 8 and FIG. 9 are conceptual diagrams further explaining a domain adaptation method based on adversarial learning according to some embodiments of this disclosure.

FIG. 10 is a flow diagram of a domain adaptation method based on adversarial learning according to some other embodiments of this disclosure.

FIG. 11 and FIG. 12 are conceptual diagrams further explaining a domain adaptation method based on adversarial learning according to some other embodiments of this disclosure.

FIG. 13 is a hardware block diagram that represents an exemplary computing device that can embody an apparatus according to diverse embodiments of this disclosure.

FIG. 14 is a block diagram of a medical image analysis system according to some embodiments of this disclosure.

### DETAILED DESCRIPTION

Desirable embodiments of this disclosure are explained in detail below by referring to the attached drawings. Merits and characteristics of embodiments disclosed and methods of achieving them would be clarified by referring to the embodiments described below with the attached drawings. However, technical idea of this disclosure is not limited to the embodiments disclosed below and can be embodied into different forms. These embodiments are simply provided to make this disclosure complete and to completely inform the scope of this invention to persons with common knowledge in the technical field of this disclosure. The technical idea of this disclosure is simply defined by the claims.

When adding reference marks to components of each figure, it must be noted that same components shall have same marks if possible, even if they appear in different figures. In addition, in explaining this disclosure, detailed explanation is omitted if specific explanation on the related disclosure or function is judged to cloud the essence of this disclosure.

If not defined otherwise, all terms used in this specification (including technical and scientific terms) can be used with meanings that can be commonly understood by persons with common knowledge in the technical field of this disclosure. Also, terms that are defined in common dictionaries are not to be interpreted ideally or excessively unless specially and clearly defined. Terms used in this specification are intended to explain embodiments, not to limit this disclosure. In this specification, singular terms include plurality unless specially mentioned in the text.

In addition, in explaining components of this disclosure, terms like first, second, etc. can be used. Such terms are only intended to discriminate the components from other components, and the essence, order, sequence, etc. of such components are not bound by these terms. If a component is described as to be “connected to,” “combined with,” or “linked to” another component, the component can be connected or linked directly to the other component, but it can also be understood as to mean that yet another component is “connected,” “combined,” or “linked” between the components.

When the terms “comprises” and/or “comprising” are used in this specification to mention components, steps, operations, and/or devices do not exclude the existence or addition of one or more other components, steps, and/or devices.

Before explaining this specification, some terms used in this specification will be clarified.

In this specification, a task refers to a problem to be solved through machine learning or a work to be performed through machine learning. For instance, if facial data are used to perform face recognition, facial expression recognition, sex classification, pose classification, etc., each of face recognition, facial expression recognition, sex classification, and pose classification can correspond to an individual domain. For another instance, if medical image data are used to recognize, classify, predict, etc. abnormality, each of abnormality recognition, classification, and prediction can correspond to an individual task. A task may also be called a target task.

In this specification, neural network is a term that embraces all types of machine learning models designed by imitating neural structures. For example, the neural network can include all types of models based on neural network such as Artificial Neural Network (ANN), Convolutional Neural Network (CNN), etc.

In this specification, instructions refer to a series of computer readable commands that constitute components of a computer program, are bound by function, and are executed by the processor.

In this specification, domain discriminator is a term that embraces models learned to discriminate domain to which certain data belong. For example, since the domain discriminator can be embodied using different types of machine learning models, technical scope of this disclosure is not limited by embodiments of the present disclosure. The domain discriminator can be called the discriminator in short.

Some embodiments of this disclosure are explained in detail below according to the attached figures.

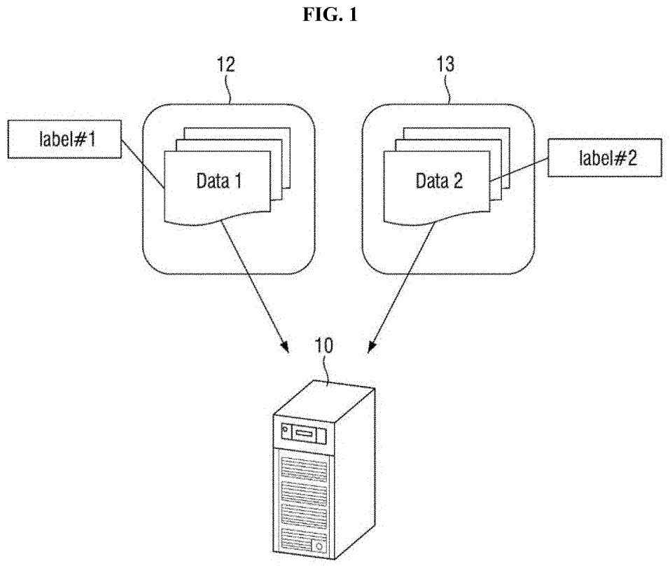

FIG. 1 is a conceptual diagram explaining a machine learning apparatus (10) and learning environment according to some embodiments of this disclosure.

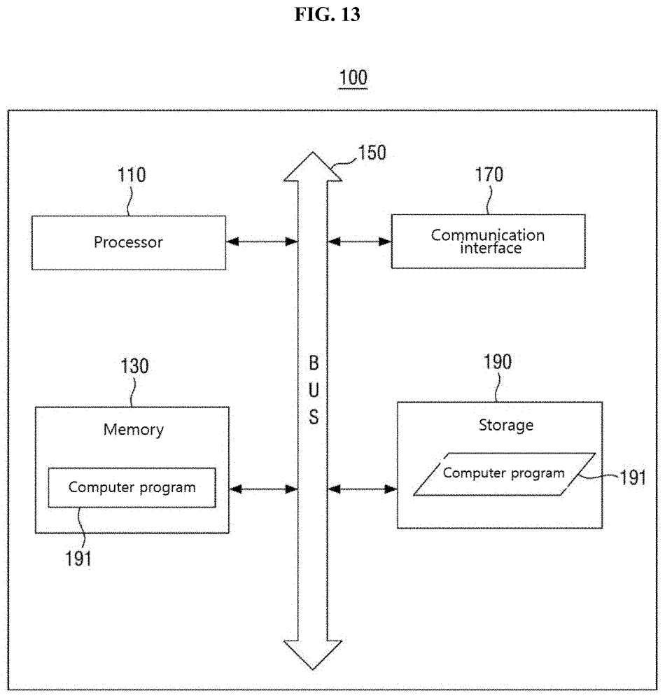

Referring to FIG. 1, the machine learning apparatus (10) is a computing device that performs machine learning on neural network. The computing device may be a laptop, desktop, server, etc., but it is not limited to these devices and can comprise all types of devices with computing functions. Refer to FIG. 13 for an example of the computing device. For convenience of explanation, the machine learning apparatus (10) will be abbreviated as the learning apparatus (10) hereafter.

FIG. 1 illustrates an example of the learning apparatus (10) embodied using a computing device, but functions of the learning apparatus (10) can be embodied using multiple computing devices in an actual physical environment. For instance, first function of the learning apparatus (10) can be embodied on a first computing device, whereas second function of the learning apparatus (10) can be embodied on a second computing device. In addition, multiple computing devices can separately embody first function and second function.

Data sets (12, 13) illustrated in FIG. 1 are the training data sets given with ground truth label, which may belong to multiple domains. For instance, the first data set (12) can be a data set comprised of multiple training samples (e.g. Data1) that belong to the first domain, and the second data set (13) can be a data set comprised of multiple training samples (e.g. Data2) that belong to the second domain different from the first domain. Here, training samples can refer to units of data for learning or diverse data. For instance, a training sample can be an image or may include diverse data other than the image depending on learning target or task.

According to diverse embodiments of this disclosure, the learning apparatus (10) can train neural network using domain adaptation based on adversarial learning. For instance, the learning apparatus (10) can construct neural network that can be utilized with the first domain and second domain using domain adaptation. Learning can be performed using the data sets (12, 13) that belong to each domain. Limited to such embodiments, the learning apparatus (10) can be named as the domain adaptation apparatus (10).

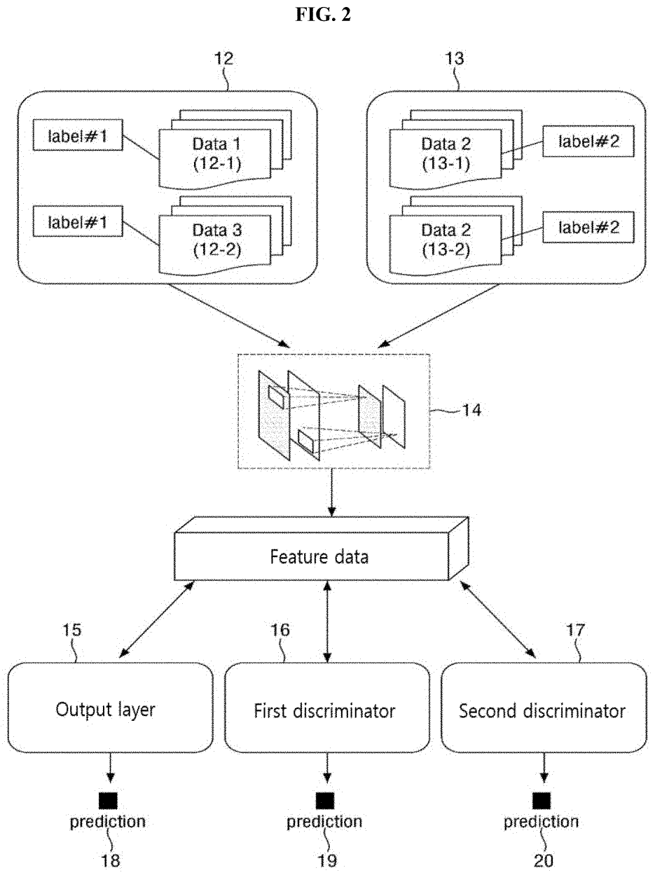

The neural network for instance can be composed as illustrated in FIG. 2. FIG. 2 exemplifies neural network that can be used to perform domain adaptation on two different domains.

As illustrated in FIG. 2, the first data set (12) that belongs to the first domain can include a data set (12-1) classified as first class and a data set (12-2) classified as second class. The second data set (13) that belongs to the second domain can include a data set (13-1) classified as the first class and a data set (12-2) classified as the second class. For convenience of understanding, domain adaptation hereafter is explained to be performed on two domains, but number of domains can change according to embodiment.

As illustrated in FIG. 2, neural network can comprise an output layer (15), two discriminators (16, 17), and shared feature extraction layer (14).

The first discriminator (16) can correspond to the first class, and the second discriminator (17) can correspond to the second class. In other words, each discriminator (16, 17) can be a class-specific discriminator. Therefore, the first discriminator (16) can be trained using the data set (12-1) that corresponds to the first class of the first domain and the data set (13-1) that corresponds to the first class of the second domain. In addition, the second discriminator (17) can be trained using the data set (12-2) that corresponds to the second class of the first domain and the data set (13-2) that corresponds to the second class of the second domain.

The output layer (15) can be trained to execute target tasks such as classification using all data sets (12, 13) that belong to the first domain and second domain.

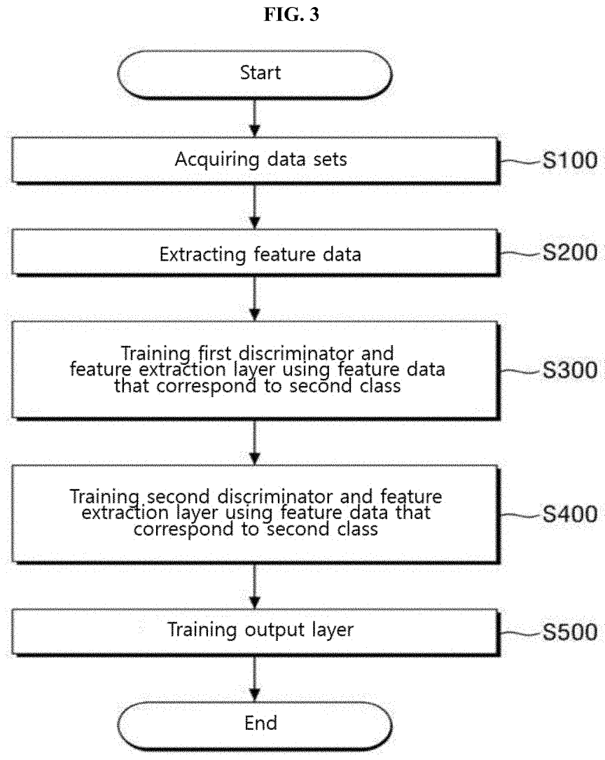

Since the feature extraction layer (14) must extract common features of the two domains, it can be trained using all data sets (12, 13) of the first domain and second domain. Here, adversarial learning can be performed between the feature extraction layer (14) and each discriminator (16, 17). In other words, the discriminator (16, 17) can be trained to discriminate domains well, and the feature extraction layer (14) can be trained to not discriminate domains well. The adversarial learning will be explained in detail by referring to FIG. 3 through FIG. 12.

FIG. 2 exemplifies neutral network that has two target classes, but number of classes can be defined and designed differently according to target task of neural network.

For instance, if the target task is a task to determine negativity, a class that indicates positive and class that indicates negative can be defined as the target classes of neural network. In addition, in this case, neural network can comprise two discriminators that respectively correspond to the two classes.

For another instance, if the target task is to diagnose cancer, a class that indicates cancer, class that indicates benign, and class that indicates normal can be defined as the target classes of neural network. In addition, in this case, neural network can include three discriminators that respectively correspond to the three classes.

For yet another instance, if the target task is to determine the type of a disease or tumor, three or more classes indicating the type of each disease or tumor can be defined as the target classes of neural network.

The learning apparatus (10) and learning environment according to some embodiments of this disclosure were explained so far by referring to FIG. 1 and FIG. 2. Methods according to diverse embodiments of this disclosure will be explained hereafter.

Each step of the methods can be executed by a computing device. In other words, each step of the methods can be embodied into one or more instructions executed by a processor of the computing device. All steps included in the methods can be executed by one physical computing device, but the methods can also be executed by multiple computing devices. For instance, the first steps of the methods can be executed by the first computing device, and the second steps of the methods can be executed by the second computing device. It is presumed hereafter that each step of the methods is executed by the learning apparatus (10) exemplified in FIG. 1. Therefore, if the subject of each operation explaining the methods is missing, they can be understood as to be executed by the exemplified apparatus (10). In addition, the methods to be described below can interchange execution order of each operation within the logically possible scope.

FIG. 3 is a flow diagram for a domain adaptation method based on adversarial learning according to some embodiments of this disclosure. However, this is only a desirable embodiment to attain the purpose of this disclosure, and some steps can be added or removed as necessary.

In the description below, it is presumed that domain adaptation is performed on two domains and that structure of neural network subject to learning is composed as exemplified in FIG. 2. However, this is only provided for convenience of understanding, and number of target domains and structure of neural network can be defined and designed differently depending on embodiments.

In S100, data sets are acquired to train neural network. For instance, the data sets can include first data set that belongs to first domain and is associated with first class, second data set that belongs to second domain and is associated with the first class, third data set that belongs to the first domain and is associated with second class, and fourth data set that belongs to the second domain and is associated with the second class. Unless mentioned otherwise, the first through fourth data sets will be used hereafter to mean the same as described.

In some embodiments, data sets of the first domain (that is, the first data set and third data set) can be composed of images generated by first shooting method, and data sets of the second domain (that is, the second data set and fourth data set) can be composed of images generated by second shooting method. In other words, domains can be classified according to the shooting method. For example, the first shooting method can be Full-Field Digital Mammography (FFDM) and the second shooting method can be Digital Breast Tomosynthesis (DBT). In this case, the neural network can be trained to execute a specific task (e.g. diagnosis of abnormality, identification of lesion) for FFDM and DBT images.

In some embodiments, data sets of the first domain (that is, the first data set or third data set) can comprise more data (that is, training samples) than data sets of the second domain (that is, the second data set or fourth data set). In this case, there can be an additional over-sampling process to increase number of samples of the second domain.

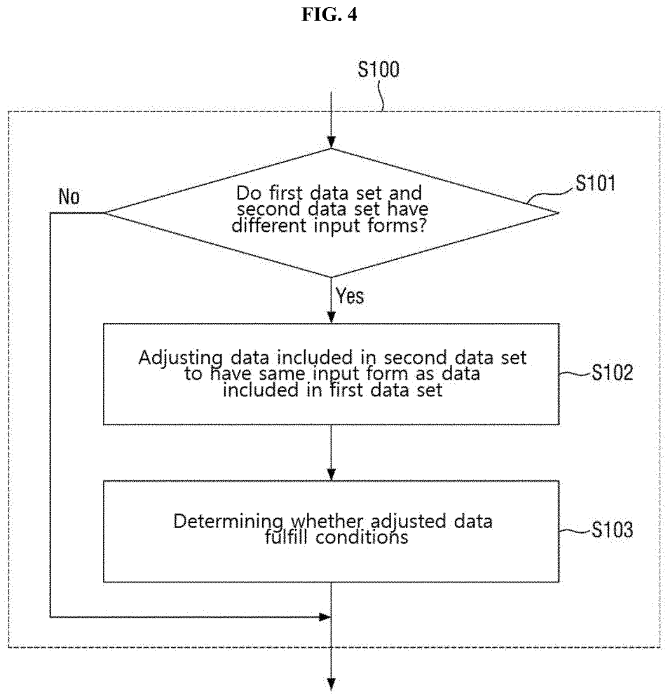

In some embodiments, data sets of the first domain (that is, the first data set and third data set) can comprise data of different forms (or formats) compared to data sets of the second domain (that is, the second data set and fourth data set). For instance, the first data set can be composed of 2D images (e.g. FFDM images) and the second data set can be composed of 3D images (e.g. DBT images). For another instance, the first data set can be composed of a single-channel or single-layer image (e.g. FFDM image) and the second data set can be composed of a multi-channel or multi-layer image (e.g. DBT image). In this case, there can be an additional process of adjusting (or transforming) form of input data according to input type of neural network before entering data into neural network. This will be explained in detail by referring to FIG. 4.

FIG. 4 assumes that input form of neural network is embodied according to the form of data sets of the first domain (that is, the first data set or third data set).

Referring to FIG. 4, S101 determines whether the first data set (or third data set) and second data set (or fourth data set) include different data forms. If data forms are different, each data included in the second data set (or fourth data set) can be adjusted (or transformed) to have same input form as the first data set (or third data set). Specific adjustment process can change according to embodiments.

In some embodiments, the first data set (or third data set) can be FFDM images and the second data set (or fourth data set) can be DBT images. DBT images can be multi-channel or D input, but neural network can be embodied to receive single-channel images as input like FFDM images. In this case, single-channel images can be extracted (or sampled) from multi-channel images to enter single-channel images into neural network as input.

In some embodiments, the first data set (or third data set) can include single-layer images and the second data set (or fourth data set) can include multi-layer images. In addition, neural network can be embodied to receive single-layer images as input. In this case, single-layer images can be extracted (or sampled) from multi-layer images to enter single-layer images extracted into neural network.

In S103 it is decided whether the adjusted data satisfy conditions. The conditions refer to criteria for determining suitability of the adjusted data as sample data for learning. For instance, the conditions can be definition, ratio of the first data set (or third data set) and second data set (or fourth data set), inclusion of certain colors, size of data, etc. The conditions can be set forth by user input or automatically according to task type. In addition, the conditions can be learned and reflected on the adjustment process.

Embodiments related to detailed process of S100 for data set acquisition were explained so far.

Explanation below is based on FIG. 3 again.

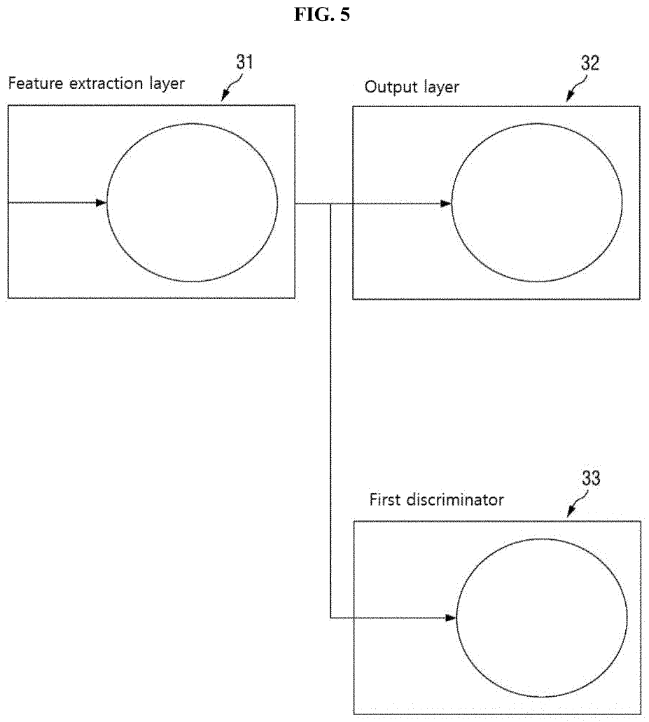

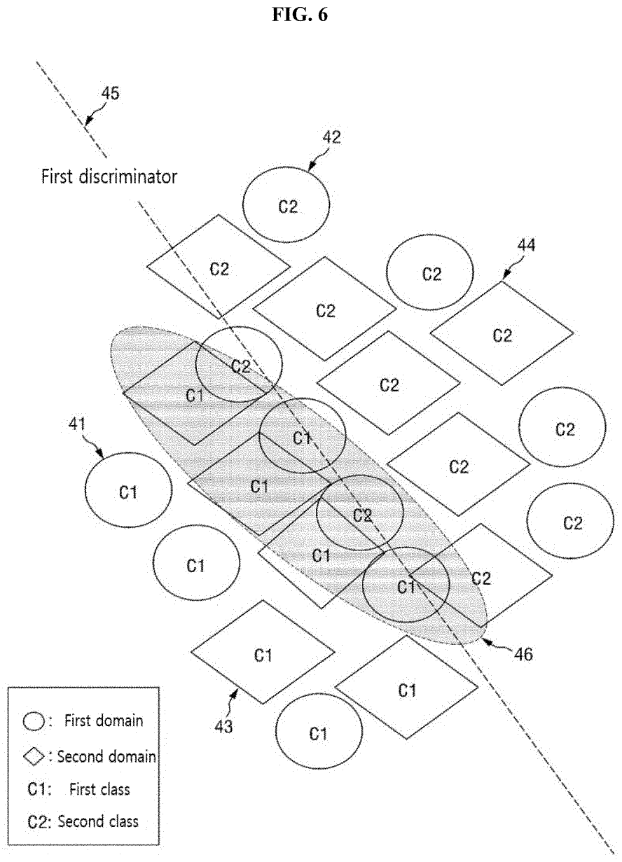

S200 through S500 below relate to process of performing domain adaptation using multiple discriminators specialized in each class. Before explaining S200 through S500, for convenience of understanding, reason for low accuracy of neural network (that is, task) that uses one discriminator will be described briefly by referring to FIG. 5 and FIG. 6.

Neural network illustrated in FIG. 5 comprises a feature extraction layer (31), output layer (32) that executes task, and one first discriminator (33) to discriminate domains. In other words, the first discriminator (33) executes an operation to discriminate domains for data sets of all classes.

When domain adaptation is performed based on adversarial learning in neural network illustrated in FIG. 5, regardless of class of data sets, the first discriminator (33) will be trained to discriminate domains well and the feature extraction layer (31) will be trained not to discriminate domains.

FIG. 6 conceptually illustrates learning result of neural network illustrated in FIG. 5. Especially, FIG. 6 conceptually illustrates distribution of each data set (41, 42, 43, 44) in feature space.

Refer to Table 1 below for meaning of each data set (41, 42, 43, 44).

TABLE 1

| | First domain | Second domain |
|---|---|---|
| First class (C1) | First data set (41) | Second data set (43) |
| Second class (C2) | Third data set (42) | Fourth data set (44) |

As illustrated in FIG. 6, if one discriminator is used, data sets (41 through 44) that belong to the two domains can be crowded in feature space regardless of class. In other words, as a result of training the feature extraction layer (31) so as to minimize difference among different domains regardless of class, distance between different classes can be reduced to have data sets of different classes (e.g. 41 and 42) mixed in crowded area (46). In this case, since reference line (45) that classifies classes cannot clearly discriminate data sets of different classes (e.g. 41 and 42), accuracy of the task is lowered. To solve the problem of lowered accuracy of the task, in some embodiments of this disclosure, multiple discriminators specialized in each class can be included in neural network. Each discriminator can correspond to one class and discriminate domains, but a certain discriminator may correspond to one or more classes depending on embodiments.

Process of performing domain adaptation using multiple discriminator is explained in detail below by referring to FIG. 3 again.

In S200, feature data of acquired data set are extracted through feature extraction layer of neural network.

In S300, the first discriminator and feature extraction layer are trained using feature data that correspond to first class. Feature data that correspond to the first class refer to feature data extracted by entering data sets of the first class (that is, the first data set and third data set) into the feature extraction layer. The first discriminator can refer to the domain discriminator in charge of the first class.

In addition, the feature extraction layer is also trained using feature data that correspond to the first class. Adversarial learning can be performed between the feature extraction layer and first discriminator.

Specific method of executing the adversarial learning can differ according to embodiments.

In some embodiments, the feature extraction layer can be trained using errors based on inverted label. The inverted label can refer to a label that inverted ground truth domain label. More specifically, domain prediction value for feature data that corresponds to the first class can be acquired by the first discriminator. The domain prediction value can refer to probability value of each domain (e.g. confidence score of each domain) indicating the domain to which the data set with extracted feature data belongs. In addition, errors can be calculated based on difference between the domain prediction value and inverted label, and weight value of the feature extraction layer can be updated by back propagation of the errors. Here, the weight value of the first discriminator is not updated by back propagation of the errors. This is because the first discriminator must be trained to discriminate domains well.

In some other embodiments, the domain prediction value of the first discriminator can be inverted, and errors can be calculated based on difference between the inverted prediction value and ground truth domain label. For instance, if probability of the first domain and second domain is respectively 8/10 and 2/10 in the domain prediction value, the inverted domain prediction value can be understood as to indicate probability of 2/10 for the first domain and 8/10 for the second domain. In addition, the weight value of the feature extraction layer can be updated by back propagation of the errors. In this case, the feature extraction layer can be trained not to discriminate domains of input data set.

In some other embodiments, errors can be calculated between the domain prediction value of the first discriminator and ground truth domain label, and gradient of the calculated errors can be inverted. In other words, the weight value of the feature extraction layer can be updated based on the inverted gradient.

In S400, second discriminator and feature extraction layer are trained using feature data that correspond to second class. Feature data that correspond to the second class refer to feature data extracted by receiving input of the data set that corresponds to the second class. The second discriminator can refer to the domain discriminator in charge of the second class.

In addition, the feature extraction layer is also trained using feature data that correspond to the second class. Adversarial learning can be performed between the feature extraction layer and second discriminator. In relation to this, refer to explanation on S300.

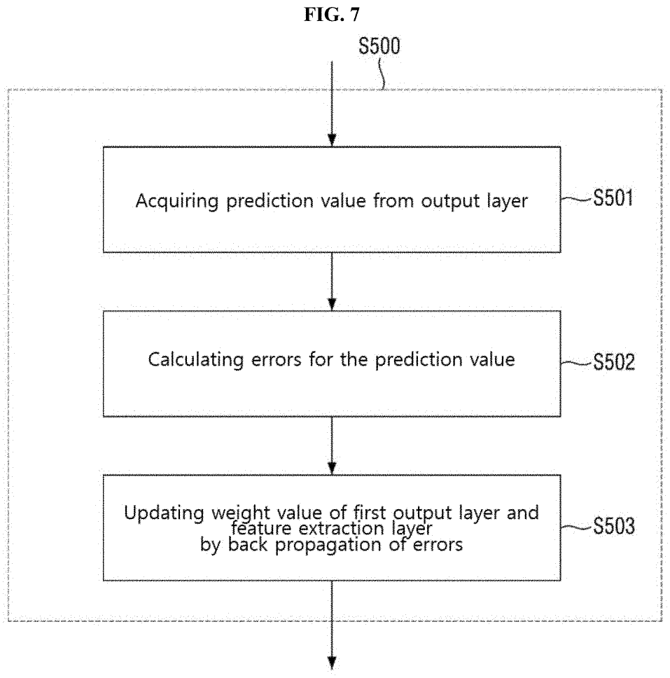

In S500, output layer is trained. The output layer is a layer trained to execute the target task (that is, task-specific layer) and outputs probability that the input data set belongs to each class (e.g. confidence score of each class). Detailed learning process of this step is illustrated in FIG. 7.

As illustrated in FIG. 7, errors about the output prediction value from the output layer (that is, difference between the prediction value and ground truth label) can be calculated, and the weight value of the output layer can be updated by back propagation of the errors calculated (S501 through S503). Here, the weight value of the feature extraction layer can be updated at the same time.

Explanation continues by referring to FIG. 3 again.

FIG. 3 illustrates that S500 is executed after S300 and S400. However, this is only to provide convenience of understanding. A part of S500 (that is, learning process associated with the first domain) can be executed together with S300, and another part (that is, learning process associated with the second domain) can be executed together with S400. Also, learning process associated with the first domain and learning process associated with the second domain can be executed at the same time.

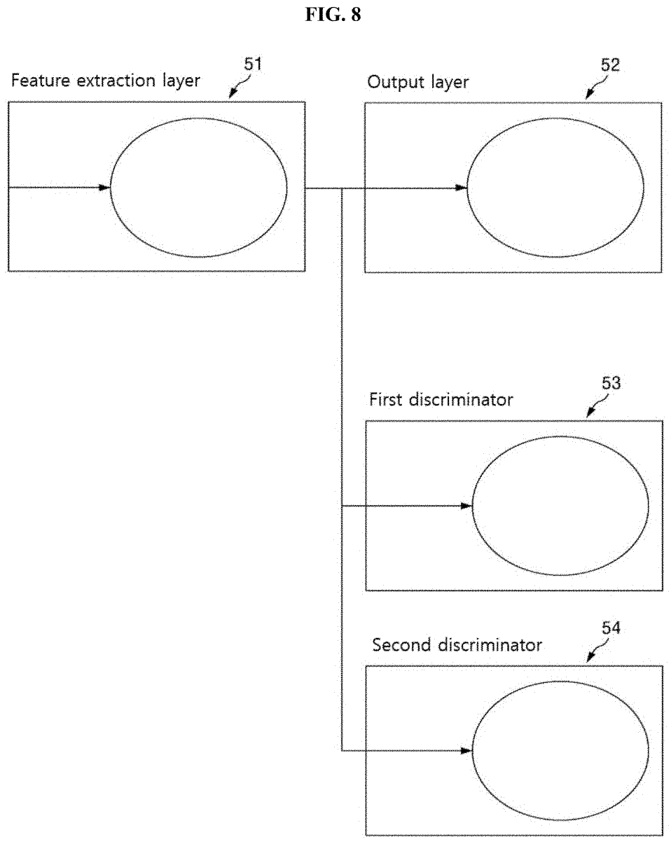

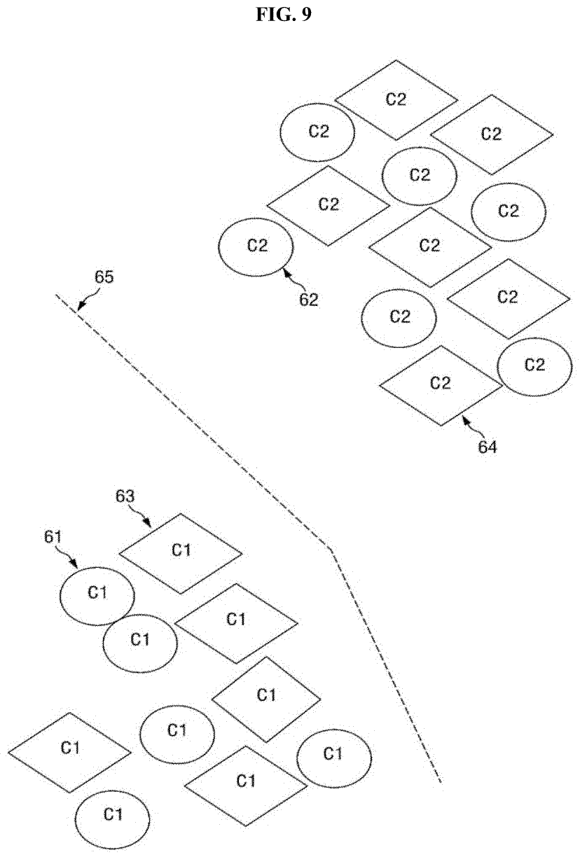

The domain adaptation method based on adversarial learning according to some embodiments of this disclosure was explained so far by referring to FIG. 3 through FIG. 7. Learning results that can be obtained by the domain adaptation method is introduced briefly below by referring to FIG. 8 and FIG. 9.

FIG. 8 exemplifies composition of neural network that applied the domain adaptation method based on adversarial learning. As exemplified in FIG. 8, the neural network can include a feature extraction layer (51), output layer, first discriminator (53) specific for first class, and second discriminator (54) specific for second class.

FIG. 9 conceptually illustrates the learning result of the neural network illustrated in FIG. 8. Especially, FIG. 9 conceptually illustrates distribution of each data set (61, 62, 63, 64) in feature space.

Refer to Table 2 below for meaning of each data set (61, 62, 63, 64).

TABLE 2

| | First domain | Second domain |
|---|---|---|
| First class (C1) | First data set (61) | Second data set (63) |
| Second class (C2) | Third data set (62) | Fourth data set(64) |

As illustrated in FIG. 9, if domain adaptation based on adversarial learning is performed using a class-specific discriminator, distance between data sets of the same class (61/63 or 62/64) can become closer in feature space, and distance between data sets of different classes (61/62 or 63/64) can become further. This is because difference between domains can be minimized for each class by performing independent adversarial learning on each class using the class-specific discriminator. In this case, since data sets of different classes are not mixed in the crowded area, the first class and second class can be classified clearly using reference line (65) that classifies classes. Therefore, accuracy of the task can be improved. In other words, the neural network can learn optimal representation that executes the task for datasets of different domains with high accuracy.

The domain adaptation method based on adversarial learning according to some embodiments of this disclosure was explained so far by referring to FIG. 3 through FIG. 9. According to the method described, the neural network that executes the task with high accuracy both in the source domain and target domain can be constructed by performing adversarial learning for each class using the class-specific discriminator. Therefore, cost of model construction in the target domain can be reduced by large.

Especially, the method described can be utilized to improve prediction performance of the neural network in a domain that cannot easily secure data (e.g. DBT domain), and the prediction performance can be improved further if the two domains have high similarity.

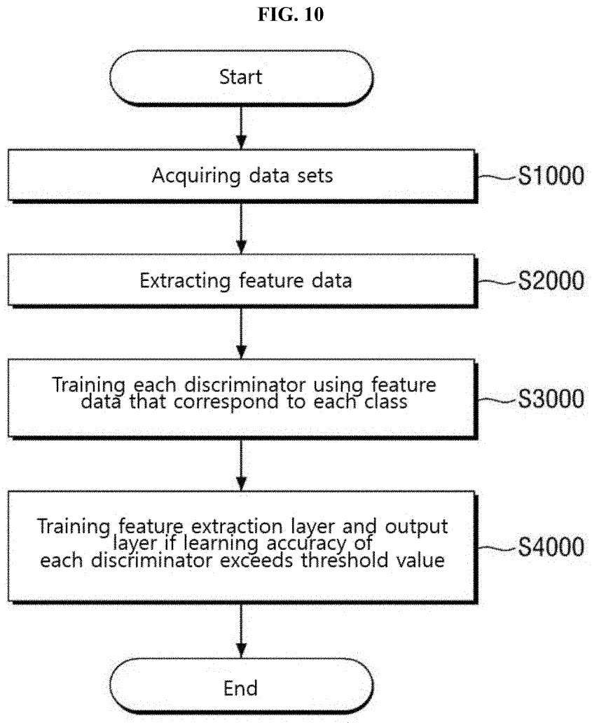

A domain adaptation method based on adversarial learning according to some other embodiments of this disclosure is explained hereafter by referring to FIG. 10 through FIG. 12.

FIG. 10 is a flow diagram of a domain adaptation method based on adversarial learning according to some other embodiments of this disclosure. For clarity of this disclosure, explanation on redundant information is to be omitted.

As illustrated in FIG. 10, in S1000 and S2000, data sets are acquired and feature data of the acquired data sets are extracted. Refer to explanation on S100 and S200 illustrated in FIG. 3 for detailed explanation on S1000 and S2000.

In S3000, each discriminator is trained using feature data that correspond to each class. In other words, the discriminator can be trained after fixing the feature extraction layer.

In S4000, when learning accuracy of each discriminator is greater than threshold value, the feature extraction layer and output layer are trained. The learning accuracy greater than the threshold value can indicate that correct answer rate of the domain prediction result of each of the discriminator is greater than the threshold value.

In S4000, adversarial learning can be performed between the feature extraction layer and each of the discriminator. In other words, unlike the discriminator, the feature extraction layer can be trained not to discriminate domains. Further explanation is omitted because method of adversarial learning was explained in detail with earlier embodiments.

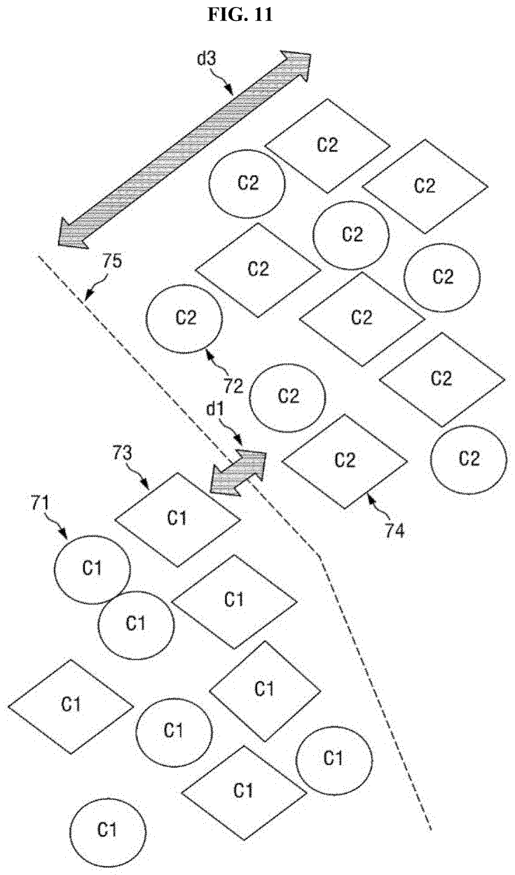

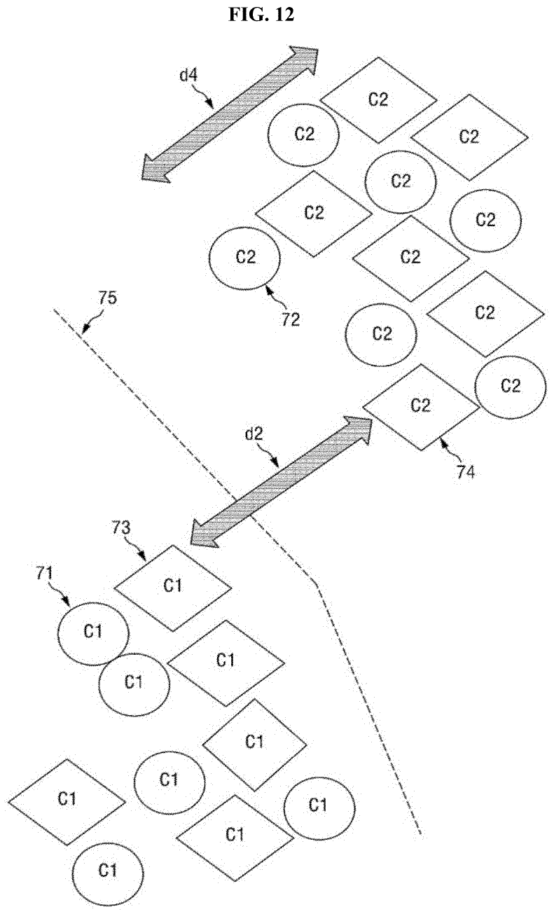

On the one hand, in some embodiments of this disclosure, adversarial learning of the feature extraction layer can be controlled based on learning accuracy of the output layer (that is, accuracy of the task). For instance, if learning accuracy of the output layer is greater than (or is greater than or equal to) the threshold value, adversarial learning can be controlled to be continued (or resumed) on the feature extraction layer. For another instance, if learning accuracy of the output layer is below (or less than or equal to) the threshold value, learning of the feature extraction layer can be controlled to stop. This is because low learning accuracy of the output layer indicates closer distance between data sets of different classes in feature space. In this case, to increase the distance between data sets of different classes, adversarial learning can be stopped and learning based on prediction errors of the output layer can be performed on the feature extraction layer. For convenience of understanding, this embodiment is further explained by referring to FIG. 11 and FIG. 12.

FIG. 11 conceptually illustrates learning result of neural network. Especially, FIG. 11 conceptually illustrates distribution of each data set (71, 72, 73, 74) in feature space. Refer to Table 3 below for meaning of each data set (71, 72, 73, 74).

TABLE 3

| | First domain | Second domain |
|---|---|---|
| First class (C1) | First data set (71) | Second data set (73) |
| Second class (C2) | Third data set (72) | Fourth data set (74) |

If domain adaptation is performed for each class, mixing of data sets of different classes (72, 73) in feature space can be prevented. Therefore, as illustrated in FIG. 11, the first class and second class can be discriminated by the reference line (75). However, if distance (d1) between data sets of different classes (72, 73) increases and distance (d3) between data sets of the same class (e.g. 72, 74) decreases, performance improvement effect of domain adaptation can be improved further. The first class and second class can be discriminated more clearly as the distance (d1) increases, and discrimination of the first domain and second domain becomes more difficult as the distance (d3) decreases.

Here, if adversarial learning using the discriminator is performed to further decrease the distance (d3), there can be a problem in which the distance (d1) also decreases in feature space depending on the case. Therefore, it is necessary to monitor the distance (d1) and control overall learning so that the distance (d1) can be increased again if necessary. A certain embodiment described can be understood as to be intended to solve this problem.

More specifically, learning accuracy of the output layer (that is, the performance evaluation result) can be used as an indicator to monitor the distance (d1). Low accuracy of the output layer can indicate closer distance (d1).

Therefore, when learning accuracy of the output layer falls below the threshold value, adversarial learning of the feature extraction layer using the discriminator can be stopped. In addition, learning of the output layer can be performed to increase the distance (d1). Learning of the output layer can include updating of the weight value of the output layer and feature extraction layer using prediction errors of the output layer.

On the contrary, when learning accuracy of the output layer becomes greater than or equal to the threshold value, adversarial learning of the feature extraction layer using the discriminator is resumed to control learning so that the distance (d3) between data sets of the same class would become closer.

In some embodiments, if learning accuracy of the output layer falls below the threshold value, the importance of the output layer increases and learning of the output layer can be performed by reflecting the increased importance. For instance, learning of the output layer can be performed by amplifying prediction errors of the output layer based on the importance. In this case, learning accuracy of the output layer can increase again.

FIG. 12 conceptually illustrates the learning result of neural network according to an embodiment.

As illustrated in FIG. 12, distance (d4) between data sets of the same class (72, 74) was decreased and distance (d2) between data sets of different classes (73, 74) was surely increased. As such, according to an embodiment described, the performance improvement effect of neural network according to domain adaptation can be maximized by controlling learning so that the distance between data sets of the same class is decreased and the distance between data sets of different classes is increased.

The domain adaptation method based on adversarial learning according to some other embodiments of this disclosure was explained so far by referring to FIG. 10 through FIG. 12. Technical idea of this disclosure explained so far by referring to FIG. 1 through FIG. 12 can be embodied by a computer readable code on a computer readable medium. The computer readable recording medium can be a removable recording medium (CD, DVD, Blu-Ray, USB drive, removable hard disk) or fixed recording medium (ROM, RAM, hard disk). The computer program recorded on the computer readable recording medium can be sent to another computing device via network such as the internet and installed and used on another computing device.

An exemplary computing device (100) that can embody the apparatus (e.g. learning apparatus 10) according to diverse embodiments of this disclosure is explained hereafter.

FIG. 13 is a hardware block diagram that represents the exemplary computing device (100).

As illustrated in FIG. 13, the computing device (100) can include one or more processors (110), bus (150), communication interface (170), memory (130) that loads a computer program (191) executed by the processor (110), and storage (190) to store the computer program (191). However, FIG. 13 only illustrates components that are related to embodiments of this disclosure. Therefore, an ordinary engineer in the technical field of this disclosure can find out that more common components can be included other than components illustrated in FIG. 13.

The processor (110) controls overall operation of each composition of the computing device (100). The processor can comprise at least one of Central Processing Unit (CPU), Micro Processor Unit (MPU), Micro Controller Unit (MCU), Graphic Processing Unit (GPU), or random processor well known in the technical field of this disclosure. In addition, the processor (110) can perform operations for at least one application or program to execute the method/operation according to embodiments of this disclosure. The computing device (100) can have one or more processors.

The memory (130) stores various data, commands and/or information. The memory (130) can load one or more programs (191) from the storage to execute the method/operation of diverse embodiments of this disclosure. The memory (130) can be embodied into a volatile memory like RAM, but the technical scope of this disclosure is not limited to this.

The bus (150) enables communication among components of the computing device. The bus (150) can be embodied into different bus forms such as address but, data bus, control bus, etc.

The communication interface (170) supports wired and wireless internet communication of the computing device (100). In addition, the communication interface (170) can support various communication methods other than the internet. For this, the interface (170) can be comprised of a communication module that is well known in the technical field of this disclosure. Depending on the case, the communication interface (170) may be omitted.

The storage (190) can non-temporarily store one or more computer programs (191), various data (e.g. learning data sets), machine learning model, etc. The storage (190) can be composed of a nonvolatile memory like flash memory, hard disk, removable disk, or random computer readable recording medium that is well known in the technical field of this disclosure.

The computer program (191) when loaded on the memory (130) can comprise one or more instructions that execute the method/operation according to diverse embodiments of this disclosure. In other words, the processor (110) can execute the methods/operations according to diverse embodiments of this disclosure by executing the one or more instructions.

For instance, the computer program (191) can comprise instructions to execute an operation that extracts feature data from multiple data sets, an operation that trains first discriminator to discriminate domain of first class using first feature data extracted from first data set that corresponds to first class of first domain among the multiple data sets, an operation that trains the first discriminator using second feature data extracted from second data set that corresponds to the first class of second domain among the multiple data sets, an operation that trains second discriminator to discriminate domain of second class using third feature data extracted from third data set that corresponds to second class of the first domain among the multiple data sets, and an operation that trains the second discriminator using fourth feature data extracted from fourth data set that corresponds to the second class of the second domain among the multiple data sets. In this case, the domain adaptation apparatus (e.g. 10) according to some embodiments of this disclosure can be embodied on the computing device (100).

The exemplary computing device (100) that can embody the apparatus according to diverse embodiments of this disclosure was explained so far by referring to FIG. 13.

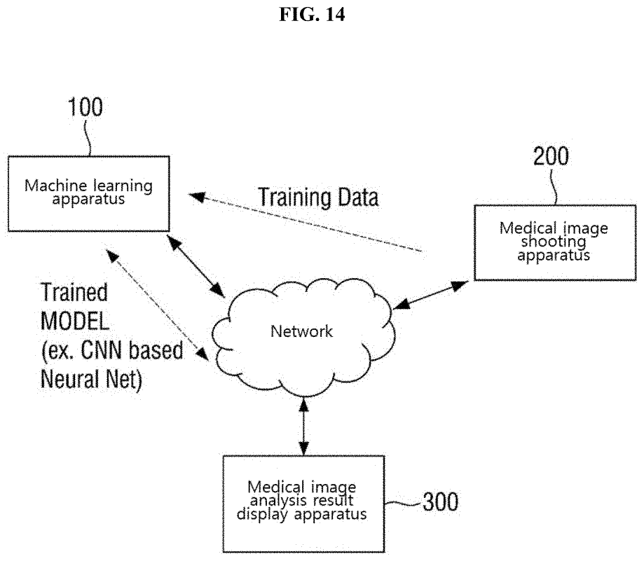

Next, composition and operation of a medical image analysis system according to some embodiments of this disclosure are explained by referring to FIG. 14.

As illustrated in FIG. 14, the medical image analysis system according to an embodiment includes a medical image shooting device (200) and machine learning device (100). According to embodiments, the medical image analysis system according to this embodiment can further comprise a medical image analysis result display apparatus (300).

The medical image shooting apparatus (200) is an apparatus that shoots medical images of body, for instance X-RAY, CT, MRI, etc. The medical image shooting apparatus (200) provides image data taken to the machine learning apparatus (100) via network. Since medical images are sensitive personal information, the network can be network that restricts connection from outside. In other words, the machine learning apparatus (100) and medical image shooting apparatus (200) can be apparatus that exist in same hospital.

The machine learning apparatus (100) in FIG. 14 can be understood as to be same as the one illustrated in FIG. 14. In other words, the machine learning apparatus (100) can accumulate image data provided by the medical image shooting apparatus (200), and, once the machine learning criteria are fulfilled, use newly accumulated image data to learn an advanced model that generates output data appropriate for the purpose of machine learning. In this process, the domain adaptation method based on adversarial learning explained by referring to FIG. 1 through FIG. 12 is executed.

Definition data of the model learned by the machine learning apparatus (100) can be sent to the medical image analysis result display apparatus (300). Unlike the medical image shooting apparatus (200) and machine learning apparatus (100), the medical image analysis result display apparatus (300) can be a computing device located outside the hospital in which the medical image shooting apparatus (200) is installed. The medical image analysis result display apparatus (300) can receive and save definition data of the model from the machine learning device (100), enter the medical image subject to analysis into the model to obtain the analysis result data, perform rending of the analysis result data, and display inference result of the medical image on screen.

Various embodiments of this disclosure and effects of such embodiments were mentioned so far by referring to FIG. 1 through FIG. 14. The effects according to the technical idea of this disclosure are not limited the effected mentioned, and other effects not mentioned can be clearly understood by ordinary engineers as described below.

The fact that all components that constitute embodiments of this disclosure are explained as to be combined or operated in combination does not mean that the technical idea of this disclosure is limited to such embodiments. In other words, within the scope of the purpose of this disclosure, all components may be operated by combining selectively into one or more groups.

Although operations are illustrated in a certain order in the drawings, it shall not be understood that the operations need to be executed according to the certain order illustrated or sequential order or that all operations illustrated need to be executed to obtain the wanted result. In certain circumstances, multi-taking and parallel processing can be advantageous. Moreover, separation of various compositions in embodiments explained shall not be understood as to mean that such separation is absolutely necessary. It shall be understood that the program components and system explained can be integrated into a single software product or packaged into multiple software products.

Embodiments of this disclosure were explained by referring to the attached drawings, but a person with common knowledge in the technical field of this disclosure can understand that this disclosure can also be embodied into other specific forms without changing the technical idea or essential characteristics. Therefore, embodiments described must be understood as to be exemplary in all aspects and not limited. The protective scope of this disclosure shall be interpreted according to the claims below, and all technical ideas within the equivalent range shall be interpreted as to be included in the scope of rights of the technical idea defined by this disclosure.

What is claimed is:

1. A domain adaptation method, executed by a computing device comprising:
extracting first feature data, second feature data, third feature data, and fourth feature data from multiple data sets by a feature extraction layer;
training a first discriminator that discriminates a domain of data corresponding to a first class using the first feature data extracted from a first data set that corresponds to a first class of a first domain among the multiple data sets;
training the first discriminator using the second feature data extracted from a second data set that corresponds to the first class of a second domain among the multiple data sets;
training a second discriminator that discriminates a domain of data corresponding to a second class using the third feature data extracted from a third data set that corresponds to a second class of the first domain among the multiple data sets;
training the second discriminator using the fourth feature data extracted from a fourth data set that corresponds to the second class of the second domain among the multiple data sets;
training the feature extraction layer and an output layer, when learning accuracy of at least one discriminator among the first discriminator and second discriminator is greater than a first threshold value; and
outputting a class classification result in which the output layer receives the first feature data, the second feature data, the third feature data and the fourth feature data, and classifies classes.

2. The method of claim 1, wherein the first domain has at least one same class as the second domain.

3. The method of claim 2, wherein the first domain and second domain are domains that include medical data sets, and wherein the class comprises at least one class among class that indicates positive or negative, class that indicates disease type and class that indicates tumor type.

4. The method of claim 1, wherein the first domain corresponds to 2D images and wherein the second domain corresponds to 3D images.

5. The method of claim 4, wherein the first data set and the third data set comprise full-field digital mammography (FFDM) images and wherein the second data set and the fourth data set comprise digital breast tomosynthesis (DBT) images.

6. The method of claim 1, wherein the first data set and the third data set comprise single-layer images and wherein the second data set and the fourth data set comprise multi-layer images.

7. The method of claim 1, wherein training the feature extraction layer and the output layer comprises updating a weight value of the feature extraction layer by back propagation of errors based on difference between inverted label that inverted ground truth domain label and a domain prediction value acquired from the first discriminator.

8. The method of claim 7, wherein updating s weight value of the feature extraction layer comprises updating a weight value of the feature extraction layer by back propagation of errors based on a difference between an inverted label that inverted ground truth domain label and a domain prediction value acquired from the second discriminator, only if learning accuracy of the output layer is greater than or equal to a second threshold value based on a learning result of the feature extraction layer.

9. The method of claim 7 further comprising:
classifying classes by receiving the first feature data, the second feature data, the third feature data and the fourth feature data and outputting class classification result in an output layer,
wherein the updating weight value of the feature extraction layer comprises increasing importance of the output layer in back propagation of the inverted label if learning accuracy of the output layer is less than a second threshold value based on a learning result of the feature extraction layer.

10. A domain adaptation apparatus comprising:
a memory that stores one or more computer-executable instructions; and
a processor configured to, by executing the stored one or more instructions:
extract first feature data, second feature data, third feature data, and fourth feature data from multiple data sets by a feature extraction layer,
train a first discriminator to discriminate a domain of data corresponding to a first class using the first feature data extracted from first data set that corresponds to a first class of a first domain among the multiple data sets,
train the first discriminator using the second feature data extracted from a second data set that corresponds to the first class of s second domain among the multiple data sets,
train a second discriminator to discriminate a domain of data corresponding to a second class using the third feature data extracted from third data set that corresponds to a second class of the first domain among the multiple data sets,
train the second discriminator using the fourth feature data extracted from a fourth data set that corresponds to the second class of the second domain among the multiple data sets,
train the feature extraction layer and an output layer when learning accuracy of at least one discriminator among the first discriminator and the second discriminator is greater than first threshold value, and
output a class classification result in which an output layer receives the first feature data, the second feature data, the third feature data and the fourth feature data, and classifies classes.

11. The apparatus of claim 10, wherein the first domain has at least one same class as the second domain.

12. The apparatus of claim 11, wherein the first domain and the second domain are domains that include medical data sets, and wherein the class comprises at least one class among class that indicates positive or negative, class that indicates a disease type, and class that indicates a tumor type.

13. The apparatus of claim 10, wherein the first domain corresponds to 2D images and the second domain corresponds to 3D images.

14. The apparatus of claim 13, wherein the first data set and the third data set comprise full-field digital mammography (FFDM) images and the second data set and the fourth data set comprise digital breast tomosynthesis (DBT) images.

15. The apparatus of claim 10, wherein the first data set and third data set comprise single-layer images and the second data set and fourth data set include multi-layer images.

16. The apparatus of claim 10, wherein a weight value of the feature extraction layer is configured to be updated by back propagation of errors based on difference between inverted label that inverted ground truth domain label and domain prediction value acquired from the first discriminator.

17. A non-transitory computer readable medium storing instructions to cause a computing device to:
extract first feature data, second feature data, third feature data, and fourth feature data from multiple data sets by a feature extraction layer;
train a first discriminator to discriminate a domain of data corresponding to a first class using the first feature data extracted from a first data set that corresponds to a first class of a first domain among the multiple data sets;
train the first discriminator using the second feature data extracted from a second data set that corresponds to the first class of a second domain among the multiple data sets;
train a second discriminator to discriminate a domain of data corresponding to a second class using the third feature data extracted from a third data set that corresponds to a second class of the first domain among the multiple data sets;
train the second discriminator using the fourth feature data extracted from a fourth data set that corresponds to the second class of the second domain among the multiple data sets;
train the feature extraction layer and an output layer when learning accuracy of at least one discriminator among the first discriminator and the second discriminator is greater than a first threshold value; and
output a class classification result in which the output layer receives the first feature data, the second feature data, the third feature data and the fourth feature data, and classifies classes.

18. The non-transitory computer readable medium of claim 17, wherein the first domain corresponds to 2D images and the second domain corresponds to 3D images.
